(19) United States
(12) Patent Application Publication
(10) Pub. No.: US 2021/0378804 A1
(43) Pub. Date: Dec. 9, 2021

(54) LIP RETRACTOR WITH INTEGRATED SUCTION

(71) Applicant: Medical University of South Carolina, Charleston, SC (US)

(72) Inventor: Walter Renne, Charleston, SC (US)

(21) Appl. No.: 16/895,915

(22) Filed: Jun. 8, 2020

(51) Int. Cl. A61C 17/10; A61B 1/24; A61C 17/08; A61C 17/06

(52) U.S. Cl. CPC A61C 17/10; A61C 17/065; A61C 17/08; A61B 1/24

(57) ABSTRACT

A lip retractor includes a first curved portion having a recess running along an outer surface, a second curved portion having a recess running along an outer surface, the second curved portion opposing the first curved portion, an arm connecting the first curved portion to the second curved portion, an interior suction chamber configured at least partially through the first and second curved portions and the arm, and a first port on an inner surface of the first curved portion, a second port on an inner surface of the second curved portion, and an evacuation connection port connected to the arm, wherein the first port, the second port and the evacuation connection port are in fluid communication with the interior suction chamber.

### BACKGROUND OF THE INVENTION

Lip retractors are commonly used during dental procedures for retraction of the mouth so that medical professionals have better access to the treatment site. In some cases, lip retractors have been combined with high volume evacuator (HVE) suction lines for expulsion of saliva. In some cases, lip retractors have been capable of attachment to a suction component. For example, conventional devices have used frontal suction combined with left and right cheek retraction for this purpose. These devices can be helpful for clearing the treatment site of saliva and for decreasing the number of biological droplets, aerosols and particulates projected from a patient's mouth. These projectiles can reach the medical professional performing the procedure, others nearby or nearby surfaces. These biological droplets, aerosols and particulates may include pathogens, virus or bacteria that could make others sick.

While conventional devices do provide some level of filtration for removing patient biological droplets, aerosols and particulates from the environment immediately surrounding the patient, conventional devices typically only provide frontal suction which is more efficient for filtering projectiles generated from lower portions of the mouth, while less effective for filtering projectiles generated from side and upper portions (lateral and superior borders) of the mouth. Better filtration mechanisms are particularly critical during the current COVID-19 pandemic to help stop the spread of coronavirus. Post-pandemic, improved filtration devices will remain useful for minimizing medical professional exposure (among others) to patient pathogens, virus or bacteria.

Thus, what is needed in the art is an improved system and device that can provide both a lip retraction and biological matter filtration mechanism which can more effectively remove patient biological droplet, aerosol and particulate projectiles generated from side and upper portions of the mouth during a dental procedure.

### SUMMARY OF THE INVENTION

In one embodiment, a lip retractor includes a first curved portion having a recess running along an outer surface; a second curved portion having a recess running along an outer surface, the second curved portion opposing the first curved portion; an arm connecting the first curved portion to the second curved portion; an interior suction chamber configured at least partially through the first and second curved portions and the arm; a first port on an inner surface of the first curved portion, a second port on an inner surface of the second curved portion, and an evacuation connection port connected to the arm, where the first port, the second port and the evacuation connection port are in fluid communication with the interior suction chamber. In one embodiment, the first curved portion and the second curved portion are substantially C-shaped. In one embodiment, the first curved portion and the second curved portion have a substantially U-shaped cross-sectional profile. In one embodiment, the lip retractor includes a contiguous wall forming the first and second curved portions, the arm, the interior suction chamber, the first and second port and the evacuation connection port. In one embodiment, the first port is part of a first plurality of ports and the second port is part of a second plurality of ports. In one embodiment, the first and second plurality of ports each include three ports. In one embodiment, at least one port is located at a top tip of at least one of the first and second curved portion. In one embodiment, at least one port is located at a bottom tip of at least one of the first and second curved portion. In one embodiment, the first and second curved portions are connected at the top and separated at the bottom. In one embodiment, the first and second curved portions are connected at the bottom and separated at the top. In one embodiment, the first and second curved portions are connected at the top and bottom. In one embodiment, the arm includes an opening. In one embodiment, a lip retractor system includes the lip; a retention bolt; and a high volume evacuator tube comprising a frame clip having an frame clip opening configured to mate with the retention bolt. In one embodiment, the retention bolt has a first threaded portion configured to interface with a second threaded portion in the frame clip opening. In one embodiment, the lip retractor system includes a high volume evacuator port connected to and in fluid communication with the evacuator tube. In one embodiment, the high volume evacuator port is configured to close or plug when not in use. In one embodiment, the lip retractor system includes a flexible tube configured to attach to and complete fluid communication between the high volume evacuator port and the evacuation connection port. In one embodiment, the lip retractor system includes a flow control valve configured to control flow between the high volume evacuator port and the evacuation connection port.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a side perspective view of a lip retractor device and high volume evacuation connector system according to one embodiment.

FIG. 2A is a front view of a lip retractor device and high volume evacuation connector system according to one embodiment with tubing connected to the evacuation connection port. FIG. 2B is a back perspective view of a lip retractor device and high volume evacuation connector system according to one embodiment.

FIGS. 3A and 3B are perspective views of a lip retractor device according to one embodiment.

FIG. 4 is a perspective view of a lip retractor device according to one embodiment.

FIGS. 5A-5J are views of a lip retractor device and high volume evacuation connector system according to one embodiment. FIG. 5A is a front perspective view thereof, FIG. 5B is an alternate front perspective view thereof, FIG. 5C is a back perspective view thereof, FIG. 5D is an alternate back perspective view thereof, FIG. 5E is a top view thereof, FIG. 5F is a bottom view thereof, FIG. 5G is a left view thereof, FIG. 5H is a right view thereof, FIG. 5I is a top view thereof, and FIG. 5J is a bottom view thereof.

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in systems and methods of lip retraction and dental aerosol filtration. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is a system and device for lip retraction with integrated suction.

Embodiments of the device provide a superior mechanism for capturing airborne patient biological matter, including droplets, aerosols and particulate projectiles generated from side and upper portions of the mouth. Rather than being a passive lip retractor, embodiments of the device actively implement suction via suction ports integrated into the lip retractor that connect to one or more suction sources. Thus, rather than having a single point of suction such as frontal suction, embodiments of the device provide for circumferential suction, providing better exposure to all areas of the month during a dental procedure.

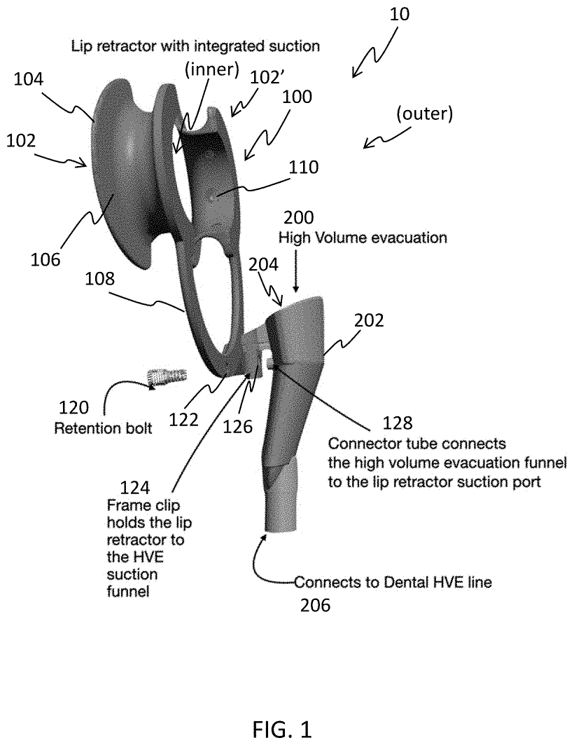

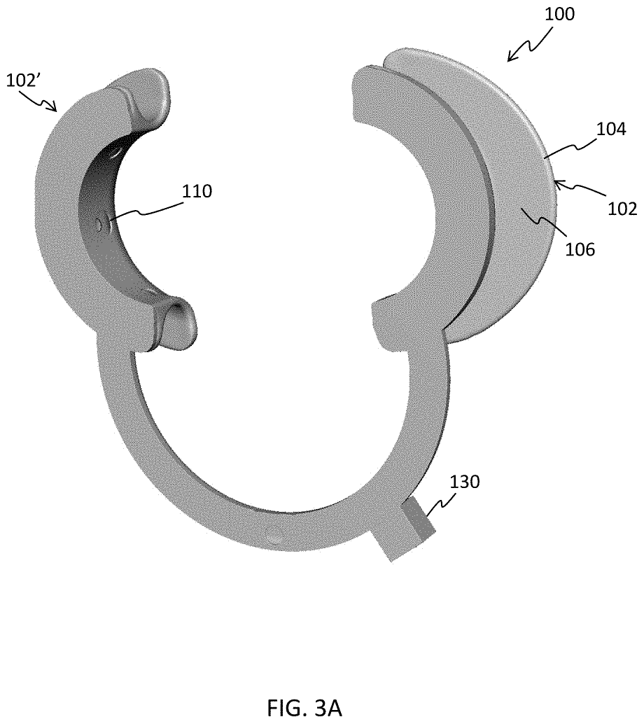

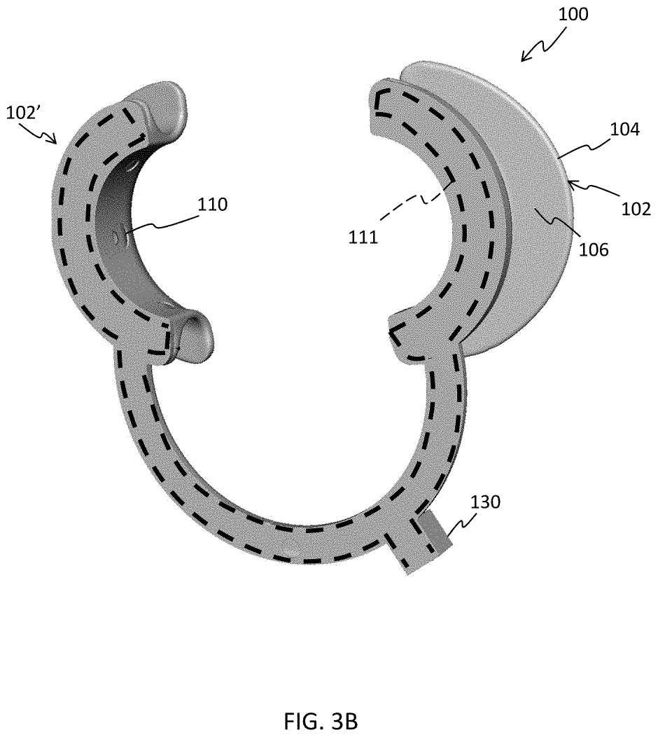

With reference now to FIG. 1, a system 10 including a lip retractor device 100 with integrated suction and a high volume evacuator 200 are shown according to one embodiment. The lip retractor device 100 includes a wall 104 forming pair of opposing curved portions 102, 102′ that are generally C-shaped curved portions, each have a recess 106 running along an outer surface of the curved portions 102, 102′. The recess 106 of each C-shaped curved portion 102, 102′ forms a U-shaped cross-sectional profile designed to partially wrap around the surface of patients lips during a dental procedure to keep them retracted. An arm 108 is attached to a lower portion of each C-shaped curved portion 102, 102′, keeping them separated and applying appropriate tension to keep lips separated and retracted during a procedure. The wall 104 forming each C-shaped curved portion 102, 102′ is contiguous to all portions of the lip retractor device 100, also forming the wall of the arm 108 and the wall of an evacuation connection port 130 extending from the arm 108. The C-shaped curved portions 102, 102′ and the arm 108 are hollow to form an interior suction chamber, which allows air and fluid to pass through it. The interior suction chamber (illustrated later in FIG. 3B) formed by the wall 104 runs through at least part of the interior of each of the C-shaped curved portions 102, 102′, the arm 108 and the evacuation connection port 130. The interior suction chamber allows air and fluid communication between suction ports 110 on the inner surface of each C-shaped curved portion 102, 102′ and the evacuation connection port 130 for removing airborne patient biological droplets, aerosols and particulate projectiles generated from various portions of the mouth, more effectively accessing side and upper portions of the mouth. Accordingly, the patient's biological matter enters the lip retractor device 100 at the suction ports 110, and moves through the C-shaped curved portions 102, 102′, down to the arm 108 and exits the lip retractor device 100 at the evacuation connection port 130.

In one embodiment, the lip retractor device 100 is connected to a high volume evacuator 200. The high volume evacuator 200 has a generally tubular structure, having a wall 202 forming a top opening 204 and a bottom connection port 206 with a interior lumen extending therebetween. The bottom connection port 206 can connect to tubing attached to a negative pressure source, such as a conventional dental high volume evacuator (HVE) line. A connection element such as a frame clip 124 can be used to connect the arm 108 of the lip retraction device 108 to the high volume evacuator 200. As shown in this embodiment, the frame clip 124 includes a threaded opening 126 that aligns with an opening at the bottom of the arm 108. A retention bolt 120 can be inserted into the opening 122 in the arm 108 and the threaded opening 126 for securing the lip retractor device 120 to the high volume evacuator. Advantageously, this embodiment combines conventional frontal suction with enhanced circumferential suction integrated into the lip retractor for filtering a much larger percentage of patient biological matter from the mouth and surrounding environment.

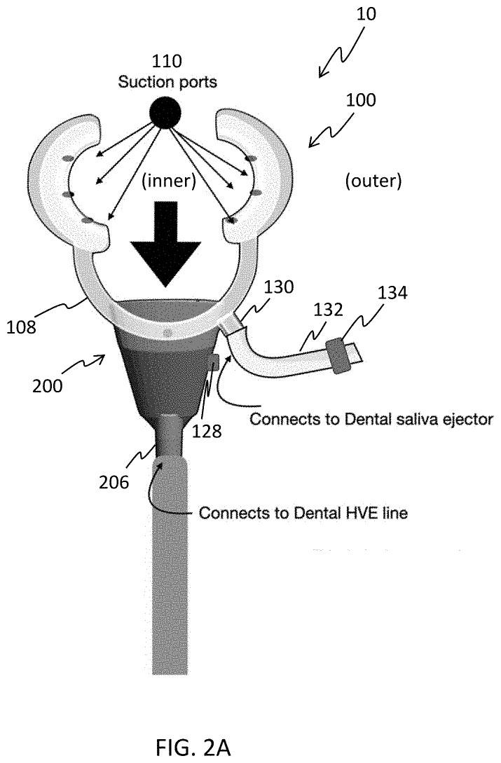

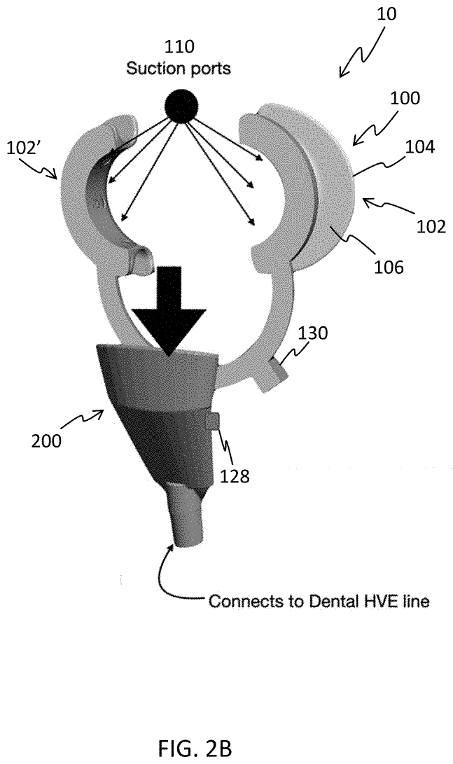

With reference now to FIGS. 2A and 2B, in one embodiment the evacuation connection port 130 can connect via tubing 132 to a negative pressure source such as a dental saliva ejector. Thus, embodiments of the system can utilize both an HVE line for frontal suction and a dental saliva ejector for lip retractor suction in increase flow dynamics and allow for greater suction power and control. Alternatively, the evacuation connection port 130 can pull suction from the HVE line by connecting the tubing 132 to the high volume evacuator 200 via high volume evacuator port 128. The high volume evacuator port 128 can be plugged when not in use. The medical provider can easily change between using one or two negative pressure sources at any time. A y-connector with shut-off valves can also be used to easily open and close connections between the lip retractor and negative pressure sources, providing an easy way to switch or modify suction levels between one and two sources. Connecting the lip retractor device 100 and the high volume evacuator 200 to separate negative pressure sources can allow for independent suction adjustment, which may change at different points in the treatment. If however only a single negative pressure source is available, the lip retractor device 100 and the high volume evacuator 200 can rely on that single negative pressure source by connecting the evacuation connection port 130 and the high volume evacuator port 128 in communication with each other. In one embodiment, at least one of the evacuation connection port 130, the high volume evacuator port 128 or the connective tubing 132 includes a flow control valve 134 to adjust the amount of suction being provided to the lip retractor device 100.

With reference now to FIGS. 3A and 3B, the lip retractor device 100 is shown in isolation according to one embodiment. The lip retractor device 100 can be 3D printed with medical grade plastics for custom builds based on patient anatomy and the procedure being performed. Suction hole 110 placement can also be customized based on these same parameters. As illustrated specifically in FIG. 3B, interior portions of the lip retractor device 100 are formed hollow to create an interior suction chamber 111 that allows air and fluid to pass from the suction ports 110 through to the evacuation connection port 130. The interior suction chamber 111 is formed from wall 104. Thus, the thickness of the wall 104 can be variable along different portions, determined by for example the desired rigidity or flexibility of the lip retractor device 100, the size of the interior suction chamber 111 at that particular portion, whether any other structures need to be accommodated for or bypassed, such as the opening 122 in the arm 108, and factors such as anatomy of the patient and the procedure being performed.

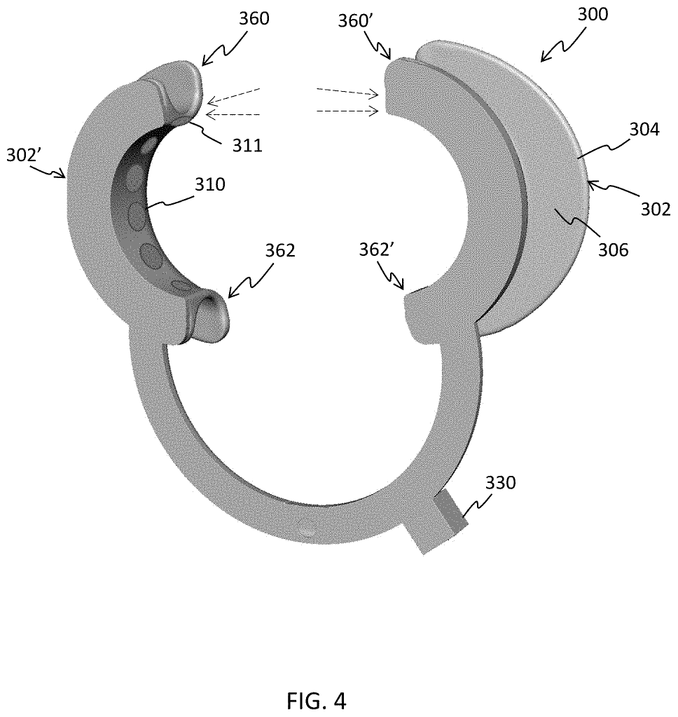

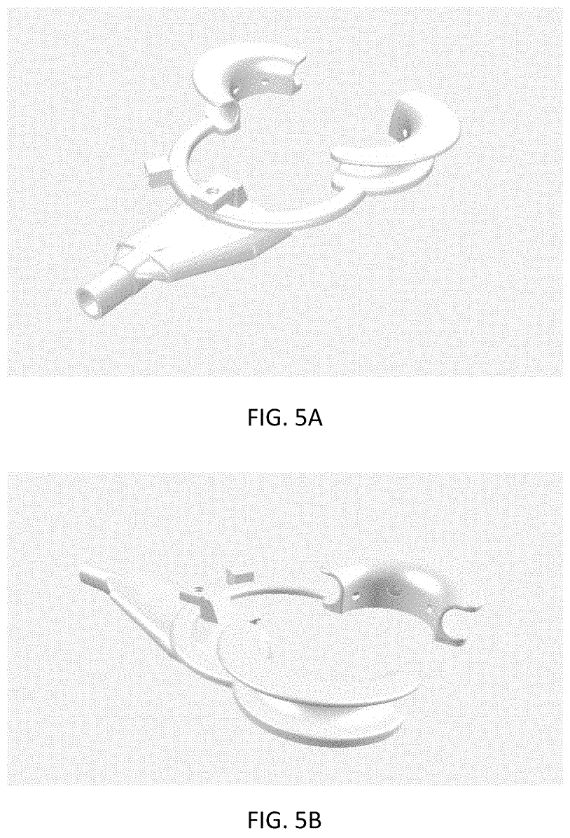

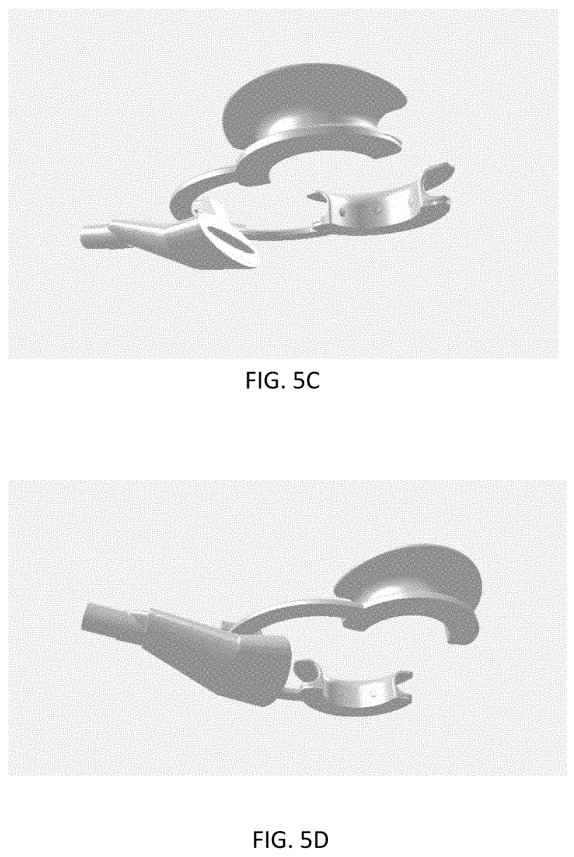

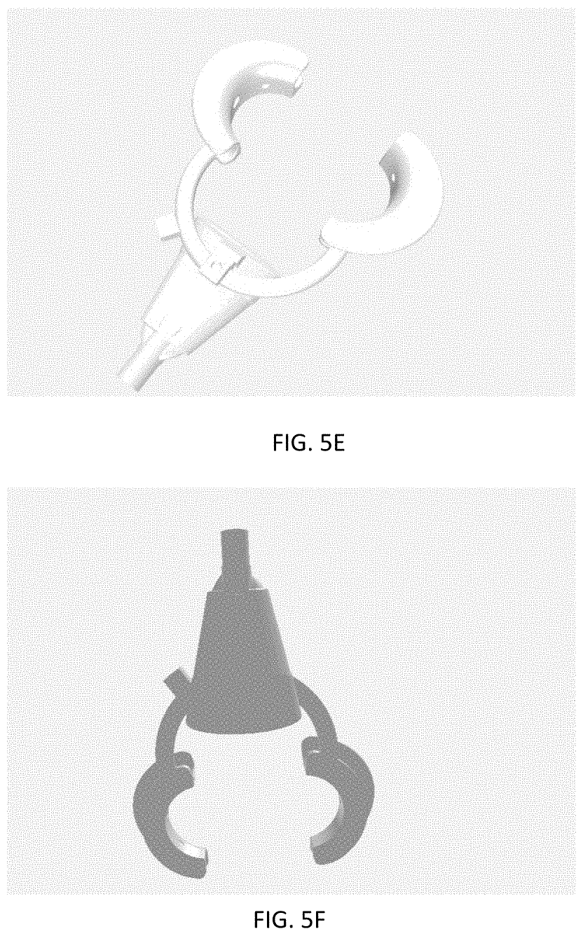

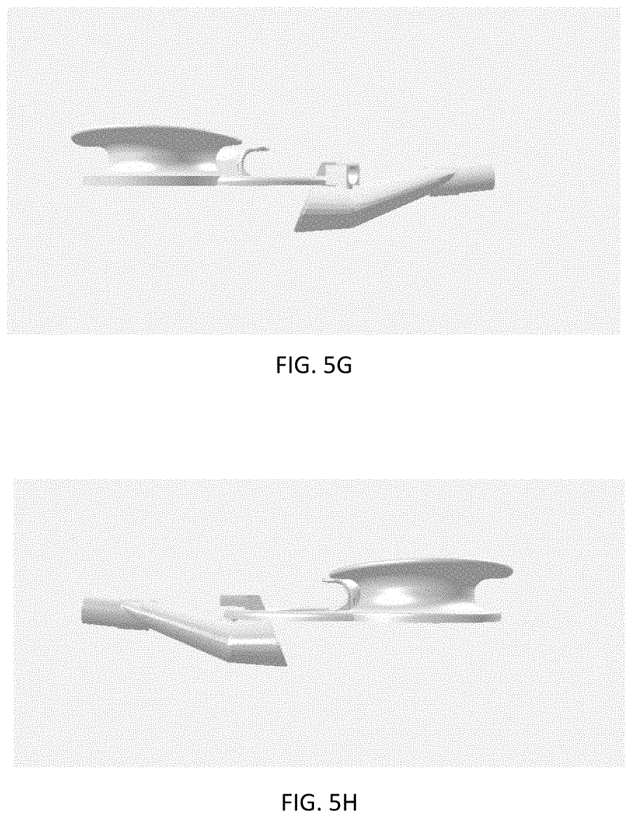

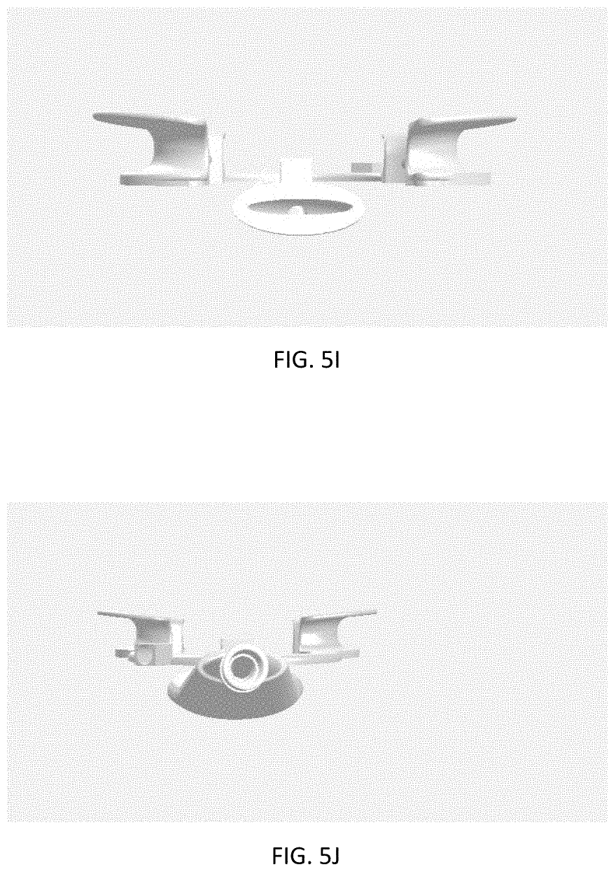

With reference now to FIG. 4, a lip retractor device 300 is shown according to one embodiment. The lip retractor device 300 includes a wall 304 forming pair of opposing C-shaped curved portions 302, 302′ that each have a recess 306. While the previous embodiment used three suction ports in each of the C-shaped curved portions, this embodiment utilizes five suction ports 310 on an inner surface of the C-shaped curved portions. As will be apparent to those having ordinary skill in the art, the number of suction ports used on the C-shaped curved portions can vary, for example including 1, 2, 3, 4, 5, 6, 7 or more suction ports per C-shaped curved portion. The geometry of the ports can also vary and do not have to be oval or circular. For example the device can include one or more elongate suction slots running some or most of the length of the inner surface the C-shaped curved portion. The length of the C-shaped curved portion may also vary, for example having top or bottom tips that come very close to touching or are actually touching, or having a gap of between 0.5 and 15 centimeters. Various embodiments can have upper and/or lower gaps of between 0.25 and 20 centimeters including gap sizes at every 0.25 centimeter interval therebetween. In one embodiment, one of the upper 360, 360′ or lower 362, 362′ tips of the C-shaped curved portions are connected and contiguous, thus presenting only one gap instead of two. In one embodiment, the entire lip retractor is connected around the entire perimeter and both the top or bottom tips of the C-shaped curved portion are connected, presenting no gaps. These embodiments (e.g. one gap or no gaps) allow for additional placement options for suction ports at top and bottom inner surfaces of the lip retractor by extending the suction chamber to those portions. As shown specifically in FIG. 4, a tip of the C-shaped curved portion (here shown at top tip 360) can feature a suction port directed towards the gap separating the opposing C-shaped curved portions. In one embodiment, the tip of the C-shaped curved portion can curve inwards to further separate a suction port located at the tip from the lips, preventing the lip from being pulled against the suction port.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A lip retractor comprising:
a first curved portion having a recess running along an outer surface;
a second curved portion having a recess running along an outer surface, the second curved portion opposing the first curved portion;
an arm connecting the first curved portion to the second curved portion;
an interior suction chamber configured at least partially through the first and second curved portions and the arm; and
a first port on an inner surface of the first curved portion, a second port on an inner surface of the second curved portion, and an evacuation connection port connected to the arm, wherein the first port, the second port and the evacuation connection port are in fluid communication with the interior suction chamber.

2. The lip retractor of claim 1, wherein the first curved portion and the second curved portion are substantially C-shaped.

3. The lip retractor of claim 1, wherein the first curved portion and the second curved portion have a substantially U-shaped cross-sectional profile.

4. The lip retractor of claim 1 further comprising:
a contiguous wall forming the first and second curved portions, the arm, the interior suction chamber, the first and second port and the evacuation connection port.

5. The lip retractor of claim 1, wherein the first port is part of a first plurality of ports and the second port is part of a second plurality of ports.

6. The lip retractor of claim 5, wherein the first and second plurality of ports each include three ports.

7. The lip retractor of claim 1, wherein at least one port is located at a top tip of at least one of the first and second curved portion.

8. The lip retractor of claim 1, wherein at least one port is located at a bottom tip of at least one of the first and second curved portion.

9. The lip retractor of claim 1, wherein the first and second curved portions are connected at the top and separated at the bottom.

10. The lip retractor of claim 1, wherein the first and second curved portions are connected at the bottom and separated at the top.

11. The lip retractor of claim 1, wherein the first and second curved portions are connected at the top and bottom.

12. The lip retractor of claim 1, wherein the arm comprises an opening.

13. A lip retractor system comprising:
the lip retractor of claim 12;
a retention bolt; and
a high volume evacuator tube comprising a frame clip having an frame clip opening configured to mate with the retention bolt.

14. The lip retractor system of claim 13, wherein the retention bolt has a first threaded portion configured to interface with a second threaded portion in the frame clip opening.

15. The lip retractor system of claim 13 further comprising:
a high volume evacuator port connected to and in fluid communication with the evacuator tube.

16. The lip retractor system of claim 15, wherein the high volume evacuator port is configured to close or plug when not in use.

17. The lip retractor system of claim 13 further comprising:
a flexible tube configured to attach to and complete fluid communication between the high volume evacuator port and the evacuation connection port.

18. The lip retractor system of claim 13 further comprising:
a flow control valve configured to control flow between the high volume evacuator port and the evacuation connection port.
